(19) United States
(12) Patent Application Publication
(10) Pub. No.: US 2020/0075144 A1
(43) Pub. Date: Mar. 5, 2020

(54) MEDICAL EXAMINATION SUPPORT APPARATUS

(71) Applicant: FUJIFILM Corporation, Tokyo (JP)

(72) Inventors: Haruyasu NAKATSUGAWA, Tokyo (JP); Tsuyoshi HIRAKAWA, Tokyo (JP); Hiroshi HIRAMATSU, Tokyo (JP)

(73) Assignee: FUJIFILM Corporation, Tokyo (JP)

(21) Appl. No.: 16/554,143

(22) Filed: Aug. 28, 2019

(30) Foreign Application Priority Data
Sep. 3, 2018 (JP) 2018-164351

(51) Int. Cl. G16H 15/00; G16H 10/60; G16H 10/40

(52) U.S. Cl. CPC G16H 15/00; G16H 30/20; G16H 10/40; G16H 10/60

(57) ABSTRACT

A medical examination support apparatus includes a medical report acquisition unit that acquires a medical report, a degree of importance determination unit that determines the degree of importance of the medical report, a delivery destination determination unit that determines the delivery destination of an alert relevant to the medical report using the degree of importance, and a delivery unit that delivers the alert to the delivery destination decided by the delivery destination decision unit. The medical examination support apparatus decides the degree of importance and the delivery destination of the medical report based on the analysis result of the medical report, and delivers the medical report.

### CROSS-REFERENCE TO RELATED APPLICATIONS

This application claims priority under 35 U.S.C §119(a) to Japanese Patent Application No. 2018-164351 filed on Sep. 3, 2018. The above application is hereby expressly incorporated by reference, in its entirety, into the present application.

### BACKGROUND OF THE INVENTION

### 1. Field of the Invention

The present invention relates to a medical examination support apparatus.

### 2. Description of the Related Art

In the medical field, in order for medical staff members, such as doctors and laboratory technicians, to smoothly advance medical practices, examinations, and the like, an integrated medical examination support apparatus allowing the results of examinations and the like to be shared by medical staff members, medical departments, or the like has been used. Medical examination apparatuses (modalities) using images, such as an endoscope apparatus, an X-ray imaging apparatus, an ultrasound diagnostic apparatus, a computed tomography (CT) apparatus, and a magnetic resonance imaging (MRI) apparatus, are also widespread, and the results of image diagnosis are also shared by the medical examination support apparatus.

In the image diagnosis, a requesting doctor such as an attending doctor sends a request for examination to an examination department, and an interpretation doctor such as a radiologist combines the findings, which are the results of the interpretation of medical images obtained by the examination, into an image diagnosis report to create a medical report. The created medical report is sent to the attending doctor, who has sent a request for examination, by the medical examination support apparatus.

In recent years, a problem has been pointed out that, even though medical reports can be shared, oversight of the medical reports that the attending doctor has not checked the medical reports occurs and accordingly the response to diagnosis, treatment, and the like for patients is delayed to aggravate the medical condition. Therefore, JP2009-075927A (corresponding to US2009/083074A1) discloses a medical report creating system that determines whether or not urgent response is required based on the degree of importance in a case where the degree of importance is set in a medical report and sends a message to a requesting doctor in a case where it is determined that urgent response is required.

### SUMMARY OF THE INVENTION

The optimal delivery destination of the message (alert) described in JP2009-075927A may differ according to the degree of importance. However, the technique disclosed in JP2009-075927A has a problem that an alert delivery destination cannot be set and accordingly an alert can be delivered only to the requesting doctor.

Therefore, it is an object of the invention to provide a medical examination support apparatus capable of delivering an alert to an optimal delivery destination according to the degree of importance.

A medical examination support apparatus of the invention comprises: a medical report acquisition unit that acquires a medical report; a degree of importance determination unit that determines a degree of importance of the medical report; a delivery destination decision unit that decides a delivery destination of an alert relevant to the medical report using the degree of importance; and a delivery unit that delivers the alert to the delivery destination decided by the delivery destination decision unit.

It is preferable that, in a case where the medical report includes text, the degree of importance determination unit determines the degree of importance of the medical report by analyzing the text of the medical report.

It is preferable that the degree of importance determination unit extracts a keyword included in the text and determines the degree of importance of the medical report using the extracted keyword.

It is preferable that the degree of importance determination unit determines the degree of importance indicating urgency by extracting the keyword indicating urgency from the text.

It is preferable that the degree of importance determination unit determines the degree of importance indicating a medical condition by extracting the keyword indicating a medical condition from the text.

It is preferable that, in a case where the medical report includes an image, the degree of importance determination unit analyzes the image and determines the degree of importance of the medical report using an analysis result of the image.

It is preferable that the degree of importance determination unit specifies a medical condition using the image and determines the degree of importance of the medical report using the specified medical condition.

It is preferable that the delivery destination decision unit decides one or a plurality of delivery destinations according to the degree of importance.

It is preferable that, in a case where a plurality of delivery destinations are decided according to the degree of importance, the delivery destination decision unit decides at least a person who has sent a request for checking of the medical report as a delivery destination.

It is preferable that, in a case where a plurality of delivery destinations are decided according to the degree of importance, the delivery destination decision unit decides a group, to which a person who has sent a request for checking of the medical report belongs, as a delivery destination.

It is preferable that the delivery unit delivers the alert relevant to presence of the medical report, the degree of importance of the medical report, and/or a deadline of the medical report.

It is preferable that the delivery unit delivers the alert including a link to the medical report.

It is preferable to further comprise a feedback unit that receives an input of feedback information relevant to determination of the degree of importance and/or decision of the delivery destination and changes a determination specification of the degree of importance and/or a decision specification of the delivery destination.

It is preferable that the feedback information is information indicating appropriateness of the determination of the degree of importance and/or appropriateness of the delivery destination.

It is preferable that the feedback unit receives the input of the feedback information in response to the alert delivered by the delivery unit.

The medical examination support apparatus of the invention can deliver an alert to the optimal delivery destination according to the degree of importance.

### BRIEF DESCRIPTION OF THE DRAWINGS

FIG. 1 is an explanatory diagram showing the configuration of a medical examination support system.

FIG. 2 is an explanatory diagram showing a client terminal provided in the medical examination support system.

FIG. 3 is a block diagram showing the configuration of the client terminal.

FIG. 4 is a block diagram showing the configuration of a medical examination support apparatus.

FIG. 5 is a block diagram showing the configuration of the medical examination support apparatus.

FIG. 6 is a diagram showing a notification list screen.

FIG. 7 is a diagram showing a notification list screen.

FIG. 8 is a diagram showing a log-in notification list screen.

FIG. 9 is a diagram showing a log-in notification list screen.

FIG. 10 is a diagram showing an unread list screen.

FIG. 11 is a block diagram showing the configuration of a medical examination support apparatus.

### DESCRIPTION OF THE PREFERRED EMBODIMENTS

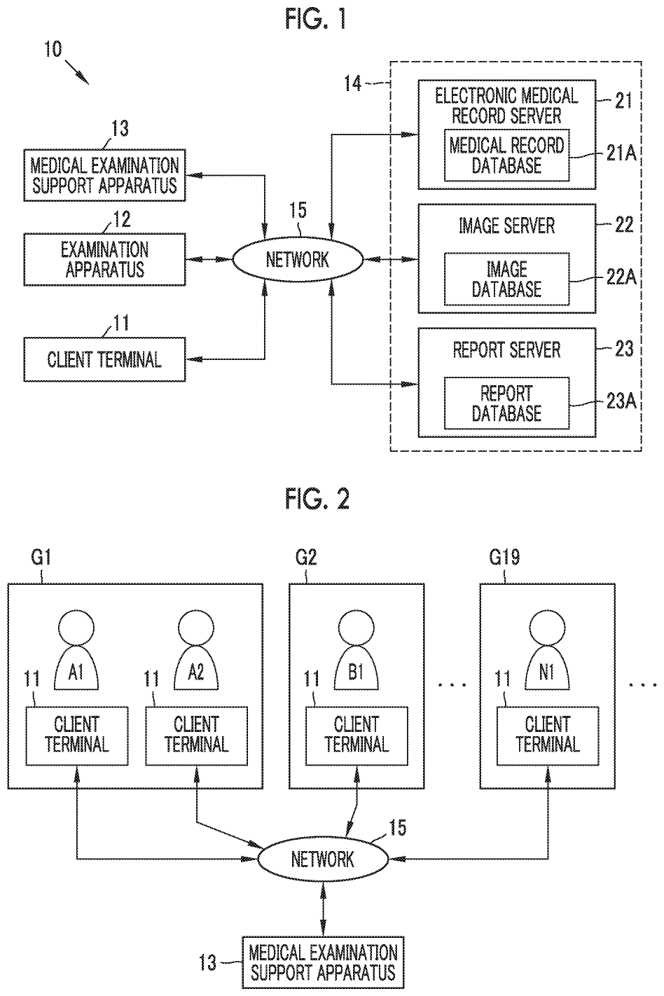

As shown in FIG. 1, a medical examination support system 10 is a computer system that performs medical examination support in a medical facility, such as a hospital, and comprises a client terminal 11, an examination apparatus 12, a medical examination support apparatus 13, and a server group 14. The respective elements configuring the medical examination support system 10 are communicably connected to each other using a network 15, such as a local area network (LAN) provided in a medical facility.

The client terminal 11 is a terminal for receiving provision of a service from the medical examination support apparatus 13 (provision of a function of the medical examination support apparatus 13), and is a computer (including a case of a tablet terminal or the like) directly operated by the medical staff member, such as a doctor, a laboratory technician, or a nurse. The client terminal 11 is installed in a medical department such as internal medicine or surgery, various examination departments such as a radiological examination department or a clinical examination department, a nurse center, or other necessary places. In addition, the client terminal 11 can be provided for each medical staff member, and can be shared by a plurality of medical staff members. Therefore, as shown in FIG. 2, the medical examination support system 10 includes a plurality of client terminals 11. For example, a group G1 is the “internal medicine” to which a doctor A1 and a doctor A2 belong, and the doctor A1 and the doctor A2 each have the client terminal 11. Similarly, for example, a group G2 is the “surgery” to which a doctor B1 belongs, and the group G2 includes at least one client terminal 11. In addition, for example, a group G19 is a “radiology department” to which a radiologist N1 belongs, and the group G19 includes at least one client terminal 11.

The examination apparatus 12 is a medical examination apparatus (modality) used for an examination. Various kinds of data including text data, such as examination conditions of the examination apparatus 12, and image data, such as acquired medical images, are stored in a server, such as an image server 22. In a case where the medical examination support apparatus 13 or the client terminal 11 requires the various kinds of data, the medical examination support apparatus 13 acquires the various kinds of data from the server. Specifically, the examination apparatus 12 includes apparatuses for acquiring medical images, such as CT, MRI, X-ray examination, ultrasound examination, and endoscope examination.

The medical examination support apparatus 13 provides the client terminal 11 with a display screen including medical examination data (for example, an image itself) and the like. The medical examination data is images, reports, and examination results acquired or created during medical practices, examinations, surgery, and the like, data obtained in the processes of other medical examinations or as results of the medical examinations, or information indicating the locations thereof (so-called link (alias) or the like).

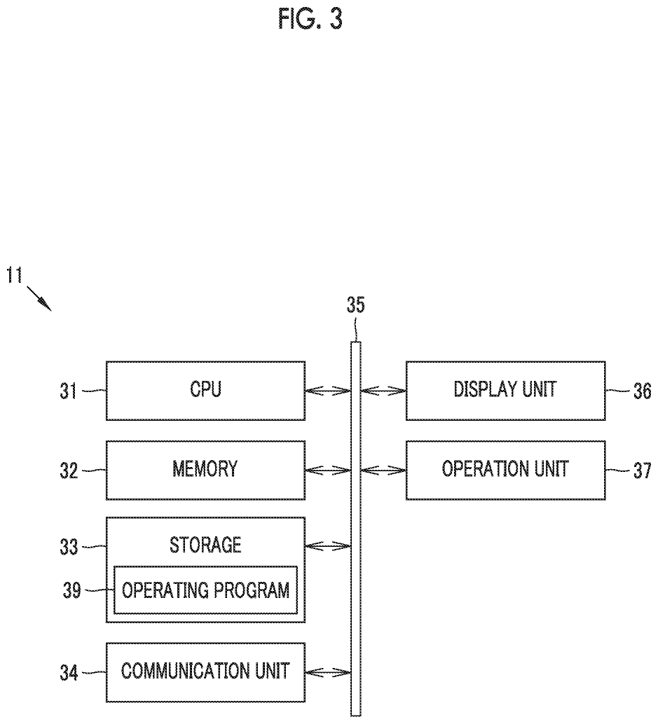

The display screen provided to the client terminal 11 by the medical examination support apparatus 13 refers to data used by the client terminal 11 to form a screen of a display unit 36 (refer to FIG. 3) of the client terminal 11. On the display screen provided to the client terminal 11 by the medical examination support apparatus 13, not only data for full screen display in which the client terminal 11 configures display of the entire screen but also data configuring display relevant to a part of the screen is included. For example, in the present embodiment, the medical examination support apparatus 13 provides the client terminal 11 with a display screen that can be displayed in a general window form in a part of the screen of the display unit 36.

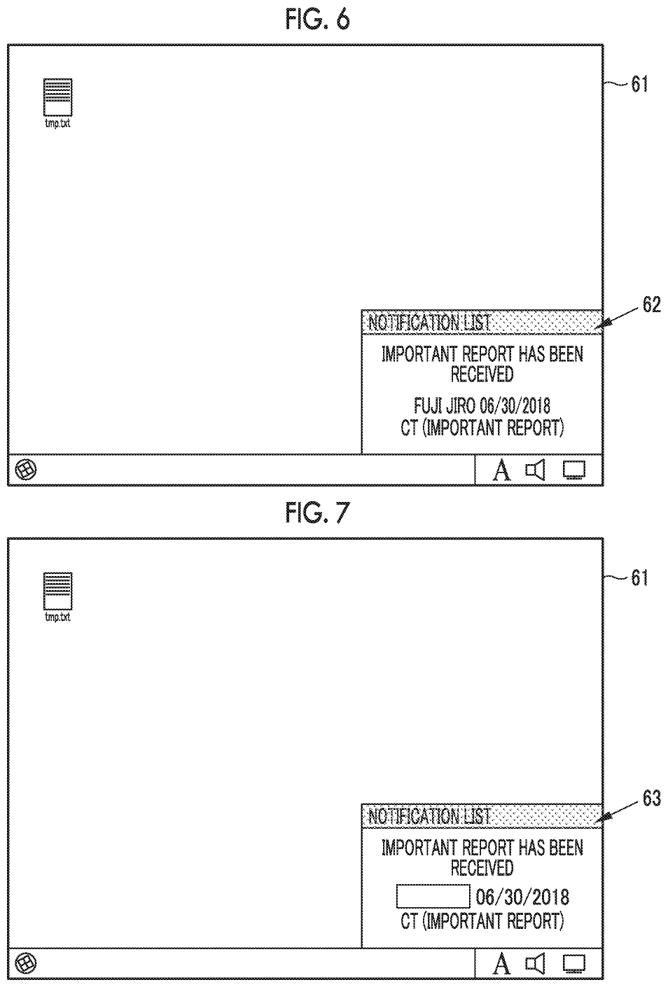

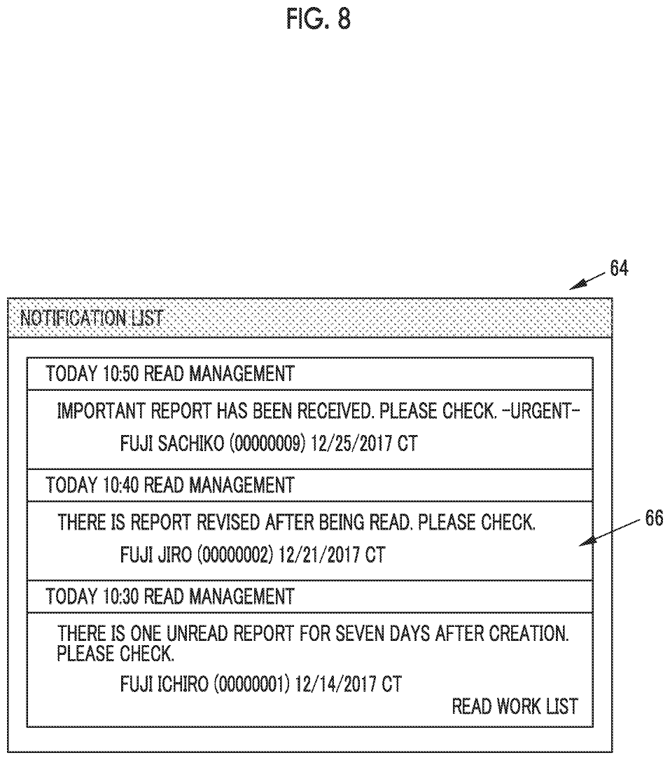

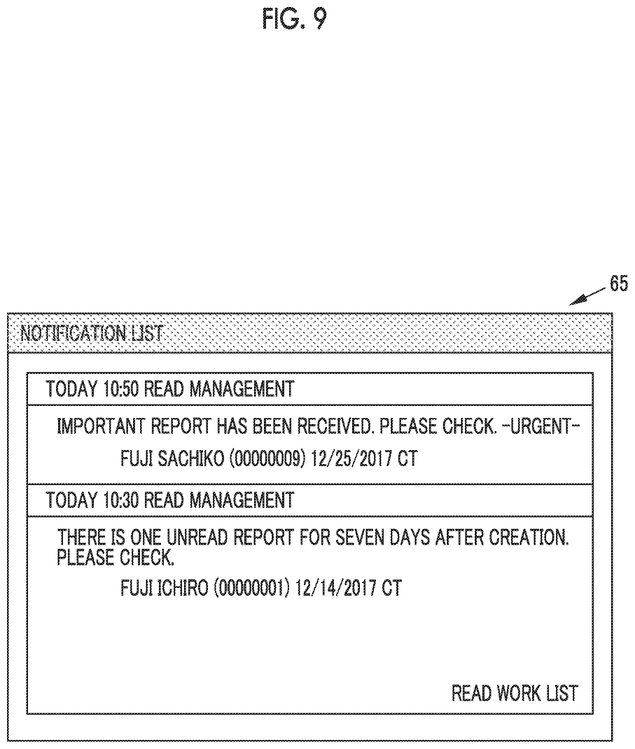

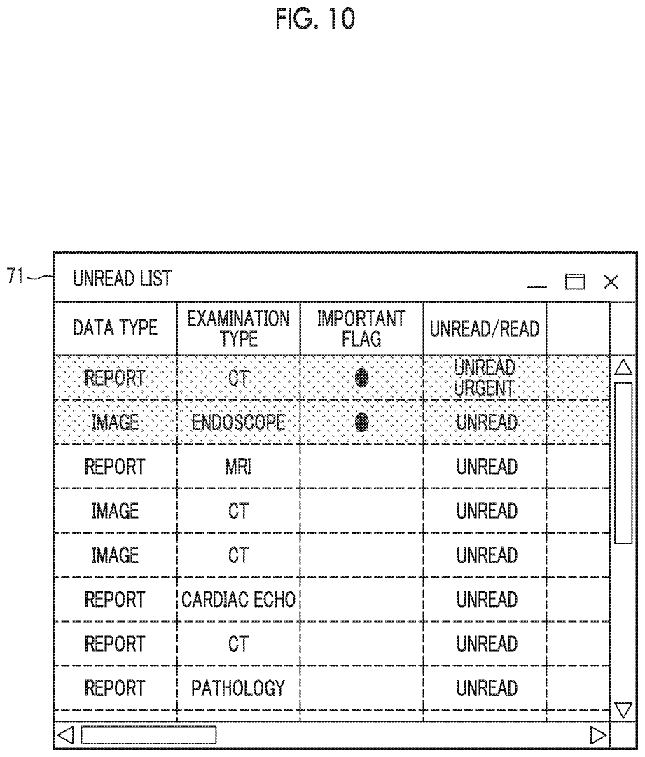

Specifically, the display screen provided to the client terminal 11 by the medical examination support apparatus 13 is notification list screens 62 and 63 (refer to FIGS. 6 and 7), log-in notification list screens 64 and 65 (refer to FIGS. 8 and 9), and an unread list screen 71 (refer to FIG. 10), and the like. The notification list screens 62 and 63 are display screens for notifying the medical staff member of the contents required for delivery of various kinds of contents to the medical staff member, generation of notes, and the like. The log-in notification list screens 64 and 65 are display screens for displaying the delivery of various kinds of contents, which have not been checked, in a case where the medical staff member logs in to the medical examination support system 10. The unread list screen 71 is a display screen for displaying the unread/read status of the delivered report, image, and the like in association with identification information of the patient. The identification information of the patient is, for example, the patient's name, date of birth, age, and sex or identification data (ID), such as a unique number and/or symbol assigned to the patient, (hereinafter, referred to as a patient ID).

The medical examination support apparatus 13 provides a display screen to the client terminal 11 in a description format using a markup language, such as an extensible markup language (XML) data, for example. The client terminal 11 displays a display screen in the XML format using a web browser. The medical examination support apparatus 13 can provide the client terminal 11 with a display screen in another format, such as JavaScript (registered trademark) object notation (JSON), instead of the XML.

The server group 14 searches for medical examination data in response to a request from the medical examination support apparatus 13, and provides the medical examination support apparatus 13 with the medical examination data corresponding to the request. The server group 14 includes an electronic medical record server 21, the image server 22, a report server 23, and the like.

The electronic medical record server 21 has a medical record database 21A in which electronic medical records are stored. The electronic medical record is a collection of one or a plurality of pieces of medical examination data. Specifically, the electronic medical records include medical examination data, such as medical practice records, results of sample examinations, patient's vital signs, orders of an examination and the like, treatment records, and accounting data, for example. The electronic medical records can be input and viewed using the client terminal 11.

The image server 22 is a so-called picture archiving and communication system (PACS) server, and has an image database 22A in which medical images are stored. The medical images are images obtained by various image examinations, such as a CT examination, an MRI examination, an X-ray examination, an ultrasound examination, and an endoscope examination. These medical images are recorded in a format based on the digital imaging and communications in medicine (DICOM) standard, for example. The medical images can be viewed using the client terminal 11.

The report server 23 has a report database 23A in which medical reports, such as an interpretation report, is stored. The medical report includes an interpretation report and a detailed examination report, and is a report in which images, numerical values, and findings obtained by examinations, such as an image examination and a subject examination, are summarized. The interpretation of a medical image and the creation of an interpretation report are performed by an interpretation doctor. A medical report can be created and/or viewed using the client terminal 11.

Each of the electronic medical record, the medical image, and the report described above includes a patient ID. In addition to the patient ID, the electronic medical record includes information for identifying the medical staff member, who has input medical examination data, for each piece of medical examination data. In addition to the patient ID, the medical image includes information for identifying the medical staff member (specifically, a laboratory technician) who has performed the examination. The report includes information for identifying the medical staff member (specifically, an interpretation doctor) who has created the report. The information for identifying the medical staff member is the name of the medical staff member or an ID, such as a unique number and/or symbol assigned to each medical staff member (hereinafter, referred to as a medical staff ID).

Each of the client terminal 11, the medical examination support apparatus 13, and the servers 21 to 23 that configure the server group 14 is configured by installing an operating system program and an application program, such as a server program or a client program, on a computer as a base, such as a server computer, a personal computer, or a workstation. That is, the basic configurations of the client terminal 11, the medical examination support apparatus 13, and the servers 21 to 23 that configure the server group 14 are the same, and each of the client terminal 11, the medical examination support apparatus 13, and the servers 21 to 23 that configure the server group 14 comprises a central processing unit (CPU), a memory, a storage, a communication unit, and a connection circuit for connecting these to each other. The communication unit is a communication interface (LAN port or the like) for connection with the network 15. The connection circuit is, for example, a motherboard for providing a system bus and/or a data bus and the like.

As shown in FIG. 3, the client terminal 11 comprises a CPU 31, a memory 32, a storage 33, a communication unit 34, a connection circuit 35, the display unit 36, and an operation unit 37. The display unit 36 is a display using, for example, liquid crystal, and has a screen for displaying at least a display screen provided by the medical examination support apparatus 13. The operation unit 37 is, for example, a pointing device such as a mouse and/or an input device such as a keyboard. The display unit 36 and the operation unit 37 can configure a so-called touch panel.

In addition to the operating system program and the like, the client terminal 11 stores an operating program 39 in the storage 33. The operating program 39 is an application program for receiving the provision of the function of the medical examination support apparatus 13 using the client terminal 11. In the present embodiment, the operating program 39 is a program of a web browser. However, the operating program 39 can be a dedicated application program for receiving the provision of the function of the medical examination support apparatus 13. The operating program 39 may include one or a plurality of gadget engines for controlling a part or entirety of the display screen provided by the medical examination support apparatus 13. The gadget engine is a subprogram that realizes various functions by being operated in conjunction with a web browser or the like.

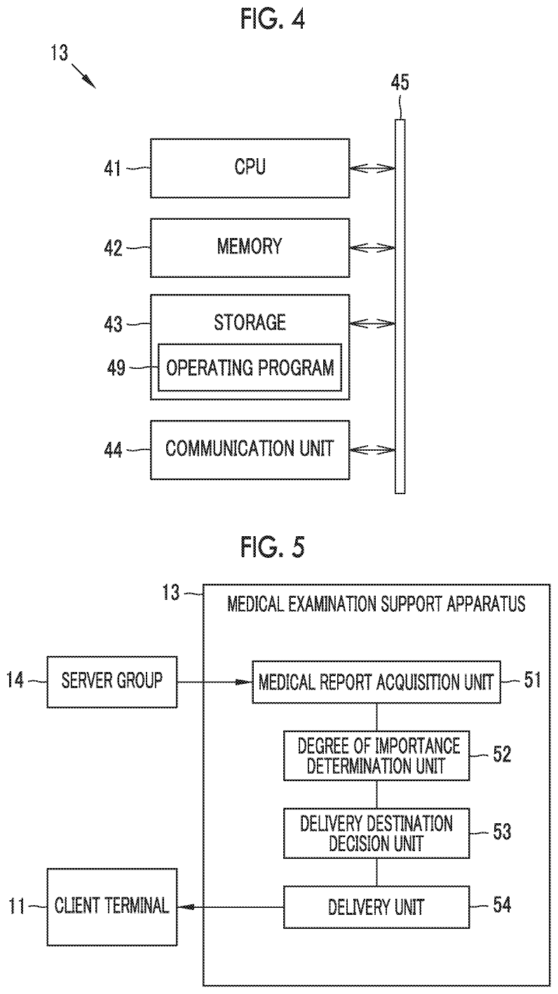

As shown in FIG. 4, the medical examination support apparatus 13 comprises a CPU 41, a memory 42, a storage 43, a communication unit 44, and a connection circuit 45. The medical examination support apparatus 13 can comprise a display unit and/or an operation unit as necessary similarly to the client terminal 11. In the present embodiment, the medical examination support apparatus 13 does not have a display unit and an operation unit although the display unit and/or the operation unit can be attached as necessary.

In the medical examination support apparatus 13, in addition to the operating system program and the like, an operating program 49 is stored in the storage 43. The operating program 49 is an application program for making a computer configuring the medical examination support apparatus 13 function as the medical examination support apparatus 13. In a case where the operating program 49 is activated, as shown in FIG. 5, the CPU 41 of the medical examination support apparatus 13 cooperates with the memory 42 to function as a medical report acquisition unit 51, a degree of importance determination unit 52, a delivery destination decision unit 53, and a delivery unit 54.

The medical report acquisition unit 51 acquires a medical report. Since medical reports are stored in the report server 23, the medical report acquisition unit 51 acquires a medical report from the report server 23. The acquisition is performed as necessary in a case where the medical examination support apparatus 13 performs processing on the medical report. For example, the medical report acquisition unit 51 acquires a medical report in a case where a new medical report is created and stored in the report server 23, in a case where the created medical report is revised, or in a case where a deadline included in the medical report is close contains a due date.

The degree of importance determination unit 52 determines the degree of importance of the medical report. The degree of importance of the medical report means the degree of necessity of urgent response to the medical report, such as in a case where a serious abnormality is pointed out in the findings based on the interpretation of the interpretation doctor. The medical report is a report of findings based on the interpretation of medical images. Therefore, in a case where an abnormality that requires urgent response to the part requested for examination is recognized as a result of the examination, the attending doctor who has sent a request can perform diagnosis or treatment on the patient quickly by checking the medical report as soon as possible. In addition, due to the interpretation of medical images, serious abnormalities may be recognized in parts other than the part requested for examination. Therefore, the degree of importance determination unit 52 automatically determines that the degree of importance of such a medical report is high, so that the degree of importance of the medical report is quickly determined. As a result, the attending doctor can check a medical report with a high degree of importance prior to other medical reports. Therefore, it is possible to prevent an accident due to the attending doctor overlooking an important medical report.

The degree of importance of the medical report is preferably the degree of importance indicating urgency and/or the degree of importance indicating a medical condition. The urgency is the degree of time indicating that the medical report needs to be checked quickly. The medical condition is the degree of content indicating that the seriousness of the medical condition described in the medical report needs to be checked as soon as possible or more carefully. The degree of importance can be divided into, for example, at least two levels of level 1 and level 2. In addition, the degree of importance can be divided into any number of levels of two or more.

Determining the degree of importance is to decide the degree of importance using information included in the medical report. The medical report includes text information, such as characters, and/or image information, such as a medical image. The degree of importance of a medical report is determined using text and/or image that is information included in the medical report.

In a case where the medical report includes text, the degree of importance determination unit 52 determines the degree of importance of the medical report by analyzing the text. The text included in the medical report includes: patient information for identifying a patient to be examined, such as a patient ID and a patient name; bibliographic information, such as a client, clinical information, an examination purpose, a part, and a modality; findings of an interpretation doctor, such as the name of a doctor in charge of interpretation, interpretation findings, and interpretation diagnosis; and the like. In addition, the text included in the medical report includes, for example, the item name of a description field, a date, and the like.

Analyzing the text means obtaining predetermined information for determining the degree of importance using a term (hereinafter, referred to as a keyword) included in the text. Therefore, analyzing the text includes extracting a keyword and using the extracted keyword. The degree of importance determination unit 52 extracts a keyword included in the text, and determines the degree of importance of the medical report using the extracted keyword. For keyword extraction, a method used in text processing can be adopted. For example, there are keyword search and morphological analysis that are machine processing of text.

Determining the degree of importance using the extracted keyword is to check, for example, the keyword itself, the expression position of the keyword, the expression frequency of the keyword, the closeness of the keyword and other keywords, and the like and decide that the degree of importance of the medical report is, for example, level 1 based on the checking result. Specifically, as the checking method, a method performed in language processing can be adopted. For example, a method, such as classification or statistics of searched keywords, and a method, such as machine learning or text mining, can be used. In addition, the text included in the medical report also includes, for example, the item name of a description field. Accordingly, in a case where the extracted keyword is the item name and its contents, it is possible to grasp the contents more precisely based on the checking result of the closeness of both keywords. As a result, it is possible to accurately determine the degree of importance.

In a case where the degree of importance is the degree of urgency for checking the medical report, the degree of importance determination unit 52 determines the degree of importance indicating urgency by extracting the keyword indicating urgency from the text. The extraction of a keyword is as described above. The keywords indicating urgency are, for example, words such as “urgent”, “immediate”, and “quickly”. Therefore, in a case where the degree of importance is divided into two levels of level 1 and level 2, a keyword is extracted from the medical report and then the extracted keyword is checked. In a case where a keyword indicating urgency is included, the medical report is determined to have the importance level 1. In a case where a keyword indicating urgency is not included, the medical report is determined to have the importance level 2. The levels can be arbitrarily divided, and it is possible to arbitrarily determine the level according to the type of keyword, the frequency of appearance of the keyword, and the like.

In a case where the degree of importance is a medical condition, the degree of importance determination unit 52 determines the degree of importance indicating the medical condition by extracting the keyword indicating the medical condition from the text. The extraction of a keyword is as described above. The medical condition includes the presence or absence of a lesion, the number of lesions, the position, size, and range (area) of a lesion, the type of lesion, or the degree of progression of a lesion. Specifically, examples of the keyword indicating a medical condition are “lesion” and “abnormality” in the case of the presence or absence of a lesion, “many” in the case of the number of lesions, parts of “lung”, “stomach”, and “large intestine” and the position name of each part in the case of the position of a lesion, “diameter”, “mm”, and “bulge” in the case of the size of a lesion, “partial” and “wide” in the case of the range of a lesion, “cancer”, “tumor”, “aneurysm”, “stenosis”, and “malignant” in the case of the type of lesion, and “progressing” and “metastasis” in the case of the degree of progression of a lesion. Therefore, in a case where the degree of importance is divided into two levels of level 1 and level 2, a keyword is extracted and then the extracted keyword is checked. For example, in a case where a plurality of keywords indicating a medical condition are included, the medical report is determined to have the importance level 1 that is the highest level of importance. In a case where a keyword indicating a medical condition is not included, the medical report is determined to have the importance level 2 that is a low degree of importance. The level can also be determined by the operation of keywords, such as the combination of keywords, the degree of proximity, and the type of item in which description occurs. The number of degrees of importance can be arbitrarily decided, and it is possible to arbitrarily determine the level of the medical report according to the type of keyword, the frequency of appearance of the keyword, and the like.

In a case where the medical report includes an image, the degree of importance determination unit 52 analyzes the image, and decides the degree of importance of the medical report using the analysis result of the image. The image included in the medical report is a medical image obtained by the examination apparatus 12. For example, the image included in the medical report is an image obtained by CT, MRI, X-ray examination, ultrasound examination, or endoscope examination.

Analyzing an image means obtaining predetermined information for determining the degree of importance as an analysis result by checking data included in the image. As a method of image analysis, a method used for image analysis of medical images can be adopted. For example, a characteristic part of the image can be extracted, the color and/or the pattern can be acquired, and the analysis result that a lesion is suspected can be obtained by comparison with the lesion. In addition, these can be performed by pattern matching, data mining, and the like using machine learning, so that the analysis result of the image can be obtained. The number of medical images provided for image analysis is not limited to one, and image analysis of two or more images may be performed. For example, by performing image analysis using images based on two different illuminations acquired for the same part, a larger amount of information for specifying a lesion can be obtained. As a result, more useful information for determining the degree of importance can be obtained as an analysis result. Using the analysis result means using information obtained as the analysis result, and the degree of importance of the medical report is determined using the analysis result.

The degree of importance determination unit 52 can specify a medical condition using the image and determine the degree of importance of the medical report using the specified medical condition. As described above, the analysis result may specify the medical condition. The medical condition is the same as that described above. In this case, the degree of importance can be determined using the specified medical condition as described above. For example, there is a case where a request for stomach CT is sent from the requesting doctor, a CT medical image is created, an interpretation doctor interprets the medical image, and no abnormality is found in the stomach, but a lung lesion is found by image analysis. Then, since an analysis result that the lesion is suspected of lung cancer is obtained, the medical report includes “suspected lung cancer” as the analysis result of the image. The degree of importance of the medical report is determined to be the highest level of importance since the analysis result includes serious contents and the doctor in charge needs to check the medical report immediately. Therefore, even in a case where the interpretation doctor overlooks an abnormal part of the lung in the medical image, the requesting doctor can prioritize the checking of the medical report since the degree of importance of the medical report is determined to be high.

The contents of the analysis and the analysis results that have been determined or decided by the degree of importance determination unit 52 are left as a history in the medical report. For example, a list of keywords used to determine the degree of importance and/or decide the delivery destination, a part of an image, a weighting result of used keywords by text mining, and the like are the history. By checking the history periodically and changing the determination specification or the decision specification, the determination or the decision can be made more accurately. In a case where it is necessary to change the determination specification or the decision specification depending on circumstances, the determination specification or the decision specification can be appropriately changed.

The analysis results of the text analysis and the image analysis may be comprehensively determined or decided with the same weighting, or may be comprehensively determined by giving a weighting to any of the analysis results. In order to prevent the medical report from being overlooked, in a case where any one of both the analysis results is an analysis result with a high degree of importance, it is preferable to use a specification adopting the degree of importance.

The delivery destination decision unit 53 decides the delivery destination of the alert relevant to the medical report using the degree of importance. Using the degree of importance includes delivering an alert in the case of a predetermined degree of importance. For example, an alert relevant to the medical report with the highest level of importance can be delivered, and no alert can be delivered for medical reports with other levels of importance. The alert relevant to the medical report is a notification regarding the medical report, and is a notification having an urgency that the delivery destination has to check the notification immediately. The method of notification can be a method that enables the delivery destination to check the notification immediately, and it is possible to use apparatuses relevant to the medical examination support apparatus 13, such as the client terminal 11 of the medical examination support apparatus 13 and a mobile terminal (not shown) at the delivery destination. For example, there are a method of sending notification by displaying an alert screen in a part of the screen of the client terminal 11 to which the delivery destination logs in, in the client terminal 11 of the medical examination support apparatus 13, and a method of sending notification by displaying an alert screen together with an alert sound on the medical examination support apparatus 13 and a mobile terminal at the delivery destination. The delivery destination is a medical staff member who needs to check the medical report. In the medical examination support apparatus 13, as described above, the medical staff member is identified by the medical staff ID. Therefore, specifically, the delivery destination can be a medical staff ID.

The delivery destination decision unit 53 decides one or a plurality of delivery destinations according to the degree of importance. Deciding a plurality of delivery destinations according to the degree of importance includes deciding a plurality of delivery destinations in order to prevent oversight for a medical report with the highest level of importance and deciding only the requesting doctor as a delivery destination for a medical report with a low level of importance. In the case of deciding a plurality of delivery destinations, a plurality of delivery destinations including different doctors in the same medical department can be decided as delivery destinations, or an associated medical staff team, for example, the entire team including an attending doctor, doctors in the same medical department, associated doctors in different departments, a medical safety management office, and nurses can be decided as delivery destinations. Therefore, for example, even in a case where the requesting doctor who has sent a request for examination creates a medical report after medical practice on the patient is completed and has no chance to check the medical report in the medical examination, it is possible to prevent the medical report from being overlooked since the medical report is viewed by another delivery destination.

In the case of deciding a plurality of delivery destinations according to the degree of importance, the delivery destination decision unit 53 decides at least a person who has sent a request for checking of the medical report as a delivery destination. For a plurality of delivery destinations, the requesting doctor, who is a person who has sent a request for checking of the medical report, is decided as a delivery destination. In general, even in a case where there are a plurality of delivery destinations, there is no ambiguity in the responsibility of the requesting doctor in charge of checking. Therefore, it is possible to prevent the requesting doctor from overlooking the medical report.

In the case of deciding a plurality of delivery destinations according to the degree of importance, the delivery destination decision unit 53 decides a group, to which a person who has sent a request for checking of the medical report belongs, as a delivery destination. The group to which a person who has sent a request for checking of the medical report belongs is, as described above, a group for each medical department. By deciding the group as a delivery destination, not only a doctor but also medical staff, such as a nurse belonging to the same medical department who can use the medical examination support apparatus 13, can know the delivery of an alert. As a result, even before the requesting doctor views the screen of the medical examination support apparatus 13, the nurse can directly notify the doctor so that it is possible to prevent an important medical report from being overlooked. In the case of deciding a plurality of delivery destinations according to the degree of importance, the delivery destination decision unit 53 decides, as a delivery destination, a group to which a person who has sent a request for checking of the medical report belongs or a person who is a person belonging to a group but is not a person who has sent a request for checking of the medical report. The person who is a person belonging to a group but is not a person who has sent a request for checking of the medical report is a doctor in the same medical department in a case where the group is each medical department. For example, even in a case where a doctor in charge is not decided for each patient, it is possible to prevent the patient's medical report from being overlooked.

The delivery unit 54 delivers an alert to the delivery destination decided by the delivery destination decision unit 53. As a delivery method, as described above, a method can be adopted in which an alert is delivered to a delivery destination. In addition to the delivery to the client terminal 11 by the medical examination support apparatus 13, in a hospital where the medical examination support apparatus 13 is installed, an alert can be delivered to a mobile terminal (not shown) since each medical staff member has a mobile terminal in many case. In a case where a mobile terminal or the like comprises a global positioning system (GPS) or in a case where a name tag or the like comprises an ID tag (not shown), such as a radio frequency identifier (RFID), it is possible to check the location of each medical staff member at the delivery destination and provide notification to a bulletin board screen (not shown) at the location.

The delivery unit delivers an alert relevant to the presence of a medical report, the degree of importance of a medical report, and/or the deadline of a medical report. The “alert relevant to the presence of a medical report” is a notification indicating that a medical report has been created, a medical report has been revised, or a medical report has been withdrawn (deleted). The “alert relevant to the degree of importance of a medical report” is a notification or display of a numerical value, a message, a symbol, and the like indicating the importance of a medical report. The “alert relevant to the deadline of a medical report” is a notification or the like including the designation of a time until which the medical report is to be viewed, and is, for example, transmission of a message indicating a request for urgent viewing. By the “alert relevant to the presence of a medical report”, notification of detailed information regarding the medical report itself is provided. By the “alert relevant to the degree of importance of a medical report ”, notification of detailed information regarding the degree of importance of a medical report is provided. In a case where the created medical report is not viewed by the “alert relevant to the deadline of a medical report”, notification can be provided to prompt the viewing. Due to these alerts, a person at the delivery destination can view the medical report at the appropriate time.

The delivery unit 54 delivers an alert including a link to the medical report. The link to the medical report is information indicating the location of the medical report, and is, for example, information of a uniform resource locator (URL). Therefore, in a case where the alert is a display screen, it is possible to access and view the medical report by executing the URL of the medical report.

The medical examination support apparatus 13 configured as described above operates as follows. First, the radiologist N1 of the radiology group G19 who is a medical staff member logs in to the medical examination support system 10 using the client terminal 11, and creates a medical report for an MRI medical image of the head whose examination has been requested by a doctor (hereinafter, referred to as a requesting doctor) A1 of the internal medicine group G1, for example. In a case where the medical report is completed, the medical report is stored in a report server. In a case where a new medical report is stored in the report server, the medical examination support apparatus 13 automatically performs the following steps. First, the medical report acquisition unit duplicates and acquires the medical report stored in the report server. Then, the degree of importance determination unit 52 determines the degree of importance using the medical report. Specifically, for example, the medical report includes an MRI image of the head and interpretation findings of the interpretation doctor, keywords of “aneurysm”, “left subdural hematoma”, and “urgent” are extracted by text analysis, and it is confirmed that there is no problem in determining the degree of urgency in the position of the keyword. In addition, an image of interest is detected in the MRI image by image analysis, and an analysis result that there is a suspicion of an aneurysm is obtained. By analyzing text and images including these, the degree of importance that is the degree of urgency is determined to be the highest level of level 1.

Then, the delivery destination decision unit 53 delivers an alert using the degree of importance of the highest level of level 1 with the requesting doctor A1 and the group G1 as delivery destinations of the alert relevant to the medical report. The alert is performed by displaying the notification list screen 62 on a part of a screen 61 of the client terminal 11 used by the requesting doctor A1, as shown in FIG. 6. The notification list screen 62 has a description that a medical report (hereinafter, referred to as an important report), which has been determined to have a high degree of importance and whose delivery destination has been decided as the requesting doctor A1, has been delivered, the name of a patient who is the target of the important report, the date of issuance of the important report, the type of modality, and a description that this is an important report. Since the notification list screen 62 includes a link, such as the URL of the important report, the important report is displayed on the screen 61 by operating the name of the patient on the notification list screen 62 by clicking or the like. For example, even in a case where the requesting doctor A1 uses the client terminal 11 to log in to the medical examination support system 10 but views a screen not related to a patient targeted by the important report, an alert is performed by displaying the notification list screen 62 shown in FIG. 6 at the lower right of the screen of the client terminal 11.

While the client terminal 11 is being used, the notification list screen 62 is always displayed in a case where the important report is delivered. Therefore, for example, in a case where there is a possibility that another patient or the like can see the notification list screen 62 during outpatient handling or the like, the notification list screen 63 can be set so as not to display patient information as shown in FIG. 7. Also on the notification list screen 63, the patient's name or the like can be displayed while clicking on a part where the patient's name or the like is not displayed. In addition, it is also possible to display the important report in the same manner as the notification list screen 62. By adopting such a configuration, the alert can be delivered to the delivery destination at any time, so that the doctor can promptly respond to the medical report.

The alert is also performed by displaying the log-in notification list screen 64 at a point in time at which the requesting doctor A1 logs in to the medical examination support system 10 using the client terminal 11, as shown in FIG. 8. The log-in notification list screen 64 is a screen on which the contents notified from the previous log-out to the current log-in are displayed collectively. The log-in notification list screen 64 displays that an important report has been received, that there is a medical report revised after being read, and that there is a medical report that has been unread for seven days. Similar to the notification list screen 62, the important report or the medical report as a target can be displayed by clicking on a patient information portion, and the patient information portion can be displayed hidden.

After checking the notified important report or medical report on the log-in notification list screen 64, the important report or medical report is recorded as read. Therefore, for example, in the case of clicking on a notification individual screen 66, on which notification of a report revised after being read is provided, to check and then logging in again, notification of the read medical report is not made on the log-in notification list screen 65, as shown in FIG. 9. In a case where it is necessary to display the read medical report, a list of read medical reports is displayed by clicking on a display screen portion of the “read work list” on the log-in notification list screen 64 or 66.

In the medical examination support apparatus 13, a person who checks the important report or the medical report to make the important report or the medical report read can be set in advance as an approver. In this case, therefore, even though a person who is not set as an approver checks the important report or the medical report, the important report or the medical report is not recorded as read. In this manner, for example, in a case where a doctor in training or the like is not set as an approver, the important report or the medical report is not recorded as read even though the doctor in training checks the important report or the medical report. In addition, for example, the important report can be recorded as read in a case where the important report is checked by two approvers. In this case, therefore, the important report remains unread in a case where the important report is checked by one approver, and is recorded as read at a point in time at which the important report is checked by another one approver thereafter. As described above, the medical examination support apparatus 13 can perform the read processing of setting the approver and recording the important report or the medical report as read. The content of the read processing can be set according to the degree of importance of the medical report. Therefore, the medical examination support apparatus 13 can deliver an alert to the optimal delivery destination according to the degree of importance.

In a case where the requesting doctor A1 uses the medical examination support system 10 using the client terminal 11, the alert is also performed by displaying that the medical report is an important report, as shown in FIG. 10. As shown in FIG. 10, on the unread list screen 71, an important flag is attached to an important report among the reports and medical data delivered to the requesting doctor A. For an important report that has not been read, in a case where there is a keyword similar to “urgent” in the above notification, this keyword is displayed. Therefore, since this can be recognized as a medical report with a higher degree of importance in the list, the important report can be checked with particular priority.

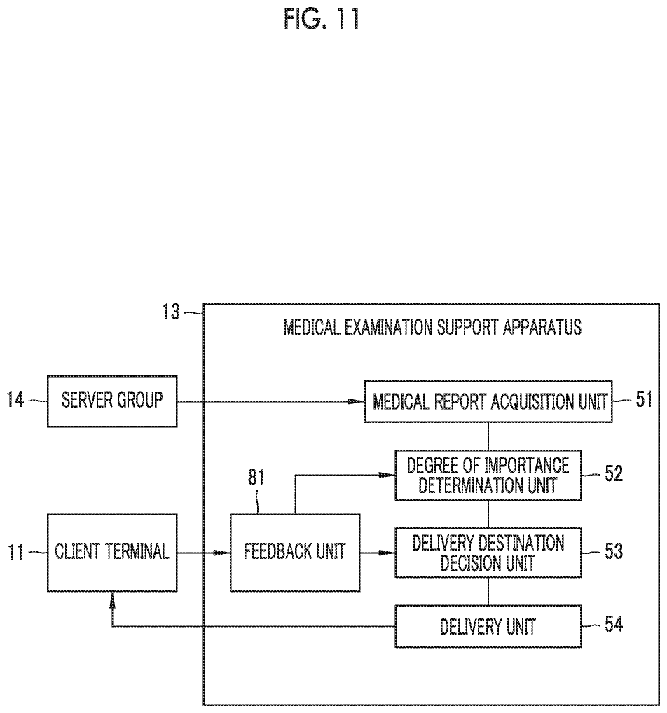

In the present embodiment, it is possible to comprise a feedback unit that receives an input of feedback information relevant to the determination of the degree of importance and/or the decision of the delivery destination and changes the determination specification of the degree of importance and/or the decision specification of the delivery destination. As shown in FIG. 11, the feedback unit 81 receives an input of feedback information.

The feedback information refers to information of a comment on the appropriateness evaluation of a person as a delivery destination of the medical report for the determination of the degree of importance and/or the decision of the delivery destination. For example, in a case where the delivery destination is not appropriate, “There is a doctor comment on automatic determination and automatic delivery” is displayed on the medical report. As the contents, feedback information, such as “Determination is appropriate, delivery to the requesting doctor A is inappropriate due to change in a person in charge, more appropriately requesting doctor B”, is input.

The feedback unit 81 receives the input of the feedback information, and changes the determination specification of the degree of importance and/or the decision specification of the delivery destination. As described above, the history of the determination specification of the degree of importance and/or the decision specification of the delivery destination is left in the medical report. Therefore, a change is made by adding the above-described comment to the history. The degree of importance determination unit 52 always sets a medical report having a comment as a learning target of text analysis and/or image analysis. Therefore, it is possible to further improve the accuracy of the determination and/or the decision.

It is preferable that the feedback unit 81 receives an input of feedback information in response to an alert delivered by the delivery unit. As described above, since the alert includes link information, such as a URL, transmission of email from the URL can be used as a reply to the alert, so that the input of feedback information can be received. As a result, since the input of feedback information can be received by one click, it is easy to collect feedback information.

By configuring the medical examination support apparatus 13 as described above, the alert is delivered to the optimal delivery destination according to the degree of importance. Therefore, since the alert is delivered to one or a plurality of persons who are most suitable for checking each medical report, it is possible to prevent the important report from being overlooked. In addition, since it is possible to prevent problems, such as delivery omissions, delivery delays, and too many delivery destinations, it is possible to prevent the important report from being overlooked. Therefore, since there is little time lag in providing notification to the delivery destination of the requesting doctor or the like after the medical report is created, it is possible to promptly respond to the medical report. In addition, since notification of medical reports is provided after the medical reports are scrutinized according to the degree of importance, the priority of the viewing of the medical reports is determined. Accordingly, since there is no need to check a large number of medical reports, it is possible to view more important medical reports without overlooking the medical reports. In addition, the degree of importance and the delivery destination are automatically determined regardless of the individual's arbitrary determination. Therefore, for example, in a case where a medical report indicates that there is a serious abnormality in another part even though the requesting doctor has recognition of a slight illness at the time of examination request, it is possible to respond to the medical report more appropriately without neglecting and overlooking the medical report. In addition, since a feedback can be given for the determination of the degree of importance and the decision of the delivery destination, it is possible to improve the accuracy of the determination and the decision.

The medical examination support system 10 has the medical examination support apparatus 13 comprising the medical report acquisition unit 51 that acquires a medical report, the degree of importance determination unit 52 that determines the degree of importance of the medical report, the delivery destination decision unit 53 that decides the delivery destination of an alert relevant to the medical report using the degree of importance, and the delivery unit 54 that delivers the alert to the delivery destination decided by the delivery destination decision unit.

The operating program 49 of the medical examination support apparatus 13 is a program causing the CPU 41 or the CPU 41 and the memory 42 to function as the medical report acquisition unit 51 that acquires a medical report, the degree of importance determination unit 52 that determines the degree of importance of the medical report, the delivery destination decision unit 53 that decides the delivery destination of an alert relevant to the medical report using the degree of importance, and the delivery unit 54 that delivers the alert to the delivery destination decided by the delivery destination decision unit.

An operation method of the medical examination support apparatus 13 comprises a medical report acquisition step in which the medical report acquisition unit 51 acquires a medical report, a degree of importance determination step in which the degree of importance determination unit 52 determines the degree of importance of the medical report, a delivery destination decision step in which the delivery destination decision unit 53 decides the delivery destination of an alert relevant to the medical report using the degree of importance, and a delivery step in which the delivery unit 54 delivers the alert to the delivery destination decided by the delivery destination decision unit.

An operation method of the medical examination support system 10 comprises a medical report acquisition step in which the medical report acquisition unit 51 acquires a medical report, a degree of importance determination step in which the degree of importance determination unit 52 determines the degree of importance of the medical report, a delivery destination decision step in which the delivery destination decision unit 53 decides the delivery destination of an alert relevant to the medical report using the degree of importance, and a delivery step in which the delivery unit 54 delivers the alert to the delivery destination decided by the delivery destination decision unit.

In the above, the hardware structures of processing units for executing various kinds of processing, such as the medical report acquisition unit 51, the degree of importance determination unit 52, the delivery destination decision unit 53, the delivery unit 54, and the feedback unit 81, are various processors shown below. The various processors include a central processing unit (CPU) that is a general-purpose processor that executes software (program) to function as various processing units, a programmable logic device (PLD) that is a processor whose circuit configuration can be changed after manufacture, such as a field programmable gate array (FPGA), and a dedicated electric circuit that is a processor having a circuit configuration that is designed for exclusive use in order to execute various kinds of processing.

One processing unit may be configured by one of various processors, or may be a combination of two or more processors of the same type or different types (for example, a combination of a plurality of FPGAs or a combination of a CPU and an FPGA). Alternatively, a plurality of processing units may be configured by one processor. As an example of configuring a plurality of processing units using one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor that realizes the function of the entire system including a plurality of processing units with one integrated circuit (IC) chip. Thus, various processing units are configured by using one or more of the above-described various processors as a hardware structure.

More specifically, the hardware structure of these various processors is an electrical circuit (circuitry) in the form of a combination of circuit elements, such as semiconductor elements.

### EXPLANATION OF REFERENCES

10: medical examination support system

11: client terminal

12: examination apparatus

13: medical examination support apparatus

14: server group

15: network

21: electronic medical record server

21A: medical record database

22: image server

22A: image database

23: report server

23A: report database

31: CPU

32: memory

33: storage

34: communication unit

35: connection circuit

36: display unit

37: operation unit

39: operating program

41: CPU

42: memory

43: storage

44: communication unit

45: connection circuit

49: operating program

51: medical report acquisition unit

52: degree of importance determination unit

53: delivery destination decision unit

54: delivery unit

61: screen

62: notification list screen

63: notification list screen

64: log-in notification list screen

65: log-in notification list screen

66: notification individual screen

71: unread list screen

81: feedback unit

What is claimed is:

1. A medical examination support apparatus, comprising:
a medical report acquisition unit that acquires a medical report;
a degree of importance determination unit that determines a degree of importance of the medical report;
a delivery destination decision unit that decides a delivery destination of an alert relevant to the medical report using the degree of importance; and
a delivery unit that delivers the alert to the delivery destination decided by the delivery destination decision unit.

2. The medical examination support apparatus according to claim 1,
wherein, in a case where the medical report includes text, the degree of importance determination unit determines the degree of importance of the medical report by analyzing the text of the medical report.

3. The medical examination support apparatus according to claim 2,
wherein the degree of importance determination unit extracts a keyword included in the text and determines the degree of importance of the medical report using the extracted keyword.

4. The medical examination support apparatus according to claim 3,
wherein the degree of importance determination unit determines the degree of importance indicating urgency by extracting the keyword indicating urgency from the text.

5. The medical examination support apparatus according to claim 3,
wherein the degree of importance determination unit determines the degree of importance indicating a medical condition by extracting the keyword indicating a medical condition from the text.

6. The medical examination support apparatus according to claim 1,
wherein, in a case where the medical report includes an image, the degree of importance determination unit analyzes the image and determines the degree of importance of the medical report using an analysis result of the image.

7. The medical examination support apparatus according to claim 6,
wherein the degree of importance determination unit specifies a medical condition using the image and determines the degree of importance of the medical report using the specified medical condition.

8. The medical examination support apparatus according to claim 1, wherein the delivery destination decision unit decides one or a plurality of delivery destinations according to the degree of importance.

9. The medical examination support apparatus according to claim 8,
wherein, in a case where a plurality of delivery destinations are decided according to the degree of importance, the delivery destination decision unit decides at least a person who has sent a request for checking of the medical report as a delivery destination.

10. The medical examination support apparatus according to claim 9,
wherein, in a case where a plurality of delivery destinations are decided according to the degree of importance, the delivery destination decision unit decides a group, to which a person who has sent a request for checking of the medical report belongs, as a delivery destination.

11. The medical examination support apparatus according to claim 1,
wherein the delivery unit delivers the alert relevant to presence of the medical report, the degree of importance of the medical report, and/or a deadline of the medical report.

12. The medical examination support apparatus according to claim 1,
wherein the delivery unit delivers the alert including a link to the medical report.

13. The medical examination support apparatus according to claim 1, further comprising:
a feedback unit that receives an input of feedback information relevant to determination of the degree of importance and/or decision of the delivery destination and changes a determination specification of the degree of importance and/or a decision specification of the delivery destination.

14. The medical examination support apparatus according to claim 13,
wherein the feedback information is information indicating appropriateness of the determination of the degree of importance and/or appropriateness of the delivery destination.

15. The medical examination support apparatus according to claim 13,
wherein the feedback unit receives the input of the feedback information in response to the alert delivered by the delivery unit.
